(19) United States
(12) Patent Application Publication
(10) Pub. No.: US 2025/0003852 A1
(43) Pub. Date: Jan. 2, 2025

(54) MICROFLUIDIC SYSTEM FOR RAPID FLUID VISCOSITY MEASUREMENT USING ACOUSTIC MICROSTREAMING

(71) Applicant: THE REGENTS OF THE UNIVERSITY OF CALIFORNIA, Oakland, CA (US)

(72) Inventors: Abraham P. Lee, Irvine, CA (US); Ruoyu Jiang, Irvine, CA (US); Abhinand Melukote Sudarsh, Irvine, CA (US); Paul Yoo, Irvine, CA (US)

(21) Appl. No.: 18/688,881

(22) PCT Filed: Sep. 21, 2022
(86) PCT No.: PCT/US22/76793

§ 371 Date: Mar. 4, 2024

Related U.S. Application Data
(60) Provisional application No. 63/247,045, filed on Sep. 22, 2021.

(51) Int. Cl. G01N 11/16; B01L 3/00

(52) U.S. Cl. CPC G01N 11/16; B01L 3/50273; B01L 2300/0816; B01L 2400/0436

(57) ABSTRACT

The present invention is directed to devices that allow for measurement of molecule/particle viscosity. The present invention features a microfluidic platform for measuring fluid viscosity. In some embodiments, the microfluidic platform may comprise a main chamber and one or more cavity acoustic transducers (CATs). The microfluidic platform may further comprise an external acoustic source coupled to the main chamber. The microfluidic platform may further comprise a fluid disposed into the main chamber. Said fluid may comprise one or more beads. The fluid may intersect the CATs to form one or more interfaces. The CATs may be configured to oscillate the interfaces to generate microstreaming flow patterns trapping the one or more beads therein. A viscosity of the fluid can be derived from the velocity.

### CROSS-REFERENCES TO RELATED APPLICATIONS

This application claims benefit of U.S. Provisional Application No. 63/247,045 filed Sep. 22, 2021, the specification of which is incorporated herein in its entirety by reference.

### STATEMENT REGARDING FEDERALLY SPONSORED RESEARCH OR DEVELOPMENT

This invention was made with government support under Grant No. IIP-1841509 awarded by NSF Center for Advanced Design and Manufacturing of Integrated Microfluidics (CADMIM). The government has certain rights in the invention.

### FIELD OF THE INVENTION

The present invention is directed to devices that allow for measurement of molecule/particle viscosity using an automated plug-and-play system with high accuracy, directly applicable to the development of medicines for the treatment of disease and cancer that requires direct injection such as antibody treatment, as well as rapid viscosity measurement of proteins/antibodies/DNA solutions for biotechnology manufacturing with very low samples.

### BACKGROUND OF THE INVENTION

The rapid measurement of fluid viscosity is an essential manufacturing step in the field of biotechnology. Sample fluids containing antibodies, proteins, and even cells have become important parts of treatments. The physical properties, including viscosity, of these biologics are critical factors in the optimization of the biomanufacturing processes.

Applications such as protein and antibody manufacturing processes require a series of preparation steps to measure their viscosities to screen and ensure quality. More importantly, the production of monoclonal antibodies as a therapeutic option requires early screening of optimal candidates based on viscosity measurements. The ability to screen functional fluids based on viscosity is a fundamental approach among industrial protocols and clinical utilities.

One of the most common approaches to measuring liquid viscosity is the falling cylinders and cone and plate viscometers. However, these instruments are bulky and require a large amount of material (>500 μL) and are very time-consuming, and can require multiple hours. A compact device that is easy to operate consumes very small amounts of fluid (<10 μL), and obtains measurements within seconds rather than hours would be beneficial to the materials and healthcare field and improve the overall efficiency of the manufacturing processes.

### BRIEF SUMMARY OF THE INVENTION

It is an objective of the present invention to provide systems that allow for measurement of molecule/particle viscosity using an automated plug-and-play system with high accuracy, directly applicable to the development of medicines for the treatment of disease and cancer that requires direct injection such as antibody treatment, as well as rapid viscosity measurement of proteins/antibodies/DNA solutions for biotechnology manufacturing with very low samples, as specified in the independent claims.

Embodiments of the invention are given in the dependent claims. Embodiments of the present invention can be freely combined if they are not mutually exclusive.

The present invention features a microfluidic platform for measuring fluid viscosity. In some embodiments, the microfluidic platform may comprise a main chamber. The main chamber may comprise an inlet. The microfluidic platform may further comprise one or more cavity acoustic transducers (CATs). The one or more CATs may be dead-end channels coupled to the main chamber. The microfluidic platform may further comprise an external acoustic source coupled to the main chamber. In some embodiments, the external acoustic source may comprise a piezoelectric transducer (PZT). The microfluidic platform may further comprise a fluid disposed through the inlet to the main chamber. Said fluid may comprise one or more beads. The fluid may intersect the CATs to form one or more interfaces. The CATs may be configured to oscillate the interfaces to generate microstreaming flow patterns trapping the one or more beads therein. A viscosity of the fluid can be derived from the velocity.

The present invention features a method for measuring fluid viscosity. In some embodiments, the method may comprise providing a microfluidic platform. The microfluidic platform may comprise a main chamber which may comprise an inlet. The microfluidic platform may further comprise one or more cavity acoustic transducers (CATs). The one or more CATs may be dead-end channels coupled to the main chamber. The method may further comprise providing an external acoustic source coupled to the main chamber. The external acoustic source may comprise a piezoelectric transducer (PZT). The method may further comprise flowing a fluid through the inlet into the main chamber. Said fluid may comprise one or more beads. The fluid may intersect the CATs to form one or more interfaces. The method may further comprise applying acoustic energy to the CATs via the external acoustic source to oscillate the interfaces. Oscillating the interfaces produces microstreaming flow patterns trapping the one or more beads therein. The method may further comprise measuring a velocity of the one or more beads in the microstreaming flow patterns. A viscosity of the fluid can be derived from the velocity.

The present invention features an acoustic microstreaming microfluidic device that achieves ultrarapid measurements of sample viscosity of less than 2 μL within seconds. First, the microfluidic well creates a cavity that forms an air-liquid interface to generate acoustic microstreaming that can trap particles and beads. Such a microfluidic well accommodates less than 2 μL of sample fluids which significantly reduces the material volume. Afterward, the speed of the acoustic microstreaming vortices is highly dependent on sample fluid viscosity and can be measured by tracking maximum beads speed near the air-liquid interface which initiates the formation of the acoustic microstreaming. For example, a higher viscosity fluid will have acoustic microstreaming that is moving slower, and a lower viscosity fluid will have acoustic microstreaming that is moving faster. More importantly, the beads' speed and trajectory will be very consistent at the air-liquid interface and can be reliably and accurately measured within 3-5 seconds to reflect and correlate the value of fluid viscosities compared to hours of bulk instruments operation time. The present invention reduces fluid consumption by 20-fold and speeds up the measurement process by thousands of folds and would be valuable and useful for extremely high throughput manufacturing of biological therapeutics and proteins.

There has not been a standardized microfluidic viscometer that can significantly shorten the measurement time, improve the accuracy of the measurement and consume an extremely low volume of sample fluid. Traditional viscometers consume a very large amount of volume in the milliliter range and take more than hours. Our demonstrated system consumes less than 2 μL of fluid and can be measured within seconds at high accuracy. This is a 20-fold reduction in volume and >4000-fold increase in throughput relative to available instruments, which significantly increases the number of candidate biologics that can be tested early in the drug discovery pipeline.

One of the unique and inventive technical features of the present invention is the implementation of cavity acoustic transducers to generate microstreaming flow patterns.

Without wishing to limit the invention to any theory or mechanism, it is believed that the technical feature of the present invention advantageously provides for measurement of a viscosity of a fluid with an extremely low sample of fluid (less than 2 μL of fluid) in a matter of seconds. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a design schematic of the microfluidic viscometer of the present invention, wherein a piezoelectric transducer is placed underneath the chip to actuate the air-liquid interface. FIG. 1B shows a real image of the actual microfluidic viscometer.

FIG. 1C shows the slow motion of beads moving within acoustic microstreaming. Size of beads=5 μm.

FIG. 2 shows a flow chart of the method for measuring the viscosity of a fluid in a microfluidic platform of the present invention.

FIG. 3A shows an 8-tip design of the present invention which allows 8 vortices to be measured within each well. FIG. 3B shows a 24-tip design which is a scaled-up version of the 8-tip design. FIG. 3C shows a circular viscometer design to induce bulk flow measurement without the need for zoomed-in magnification of acoustic microstreaming movement. FIG. 3D shows a 16 microfluidic viscometer array version of the present invention to allow multiple sample measurements. FIG. 3E shows an embodiment of the present invention implementing height-based measurement of bulk flow. FIG. 3F shows an embodiment of the present invention implementing a laddered capillary burst pressure approach to bulk flow measurement.

FIG. 4A shows a graph of maximum acoustic streaming speed under 4 Vₚₚ as an indicator to distinguish and measure viscosities of different compositions of glycerol-water mixtures. FIG. 4B shows a graph of maximum acoustic streaming speed under 8 Vₚₚ. FIG. 4C shows a graph of maximum acoustic streaming speed under 12 Vₚₚ.

### DETAILED DESCRIPTION OF THE INVENTION

Following is a list of elements corresponding to a particular element referred to herein:
100 microfluidic platform
110 main chamber
115 inlet
130 cavity acoustic transducers (CATs)
140 external acoustic source
150 interface
160 fluid
165 beads
170 microstreaming flow patterns

As used herein, Cavity Acoustic Transducers (CATs) are simple on-chip actuators that are easily fabricated and can be actuated using a battery-operated portable electronics platform. CATs are dead-end channels that are in the same plane laterally with respect to the microchannels. In some embodiments, the CATs require no additional fabrication steps other than those needed to produce a single-layer or multilayer device. When the device is filled with liquid, CATs trap bubbles creating an interface that can be excited using an external acoustic source such as a piezoelectric transducer. The interface generated by an LCAT may be comprise a gas-liquid interface, a liquid-liquid interface, a lipid membrane, a polymer membrane, a nano-particle membrane, or a combination thereof. In some embodiments, the liquid-liquid interface may comprise a plurality of immiscible liquids. As used herein, the term “immiscible liquids” refers to a set of liquids that are incapable of mixing (e.g. water and a hydrophobic liquid such as oil). In other embodiments, the liquid-liquid interface may comprise a thin physical barrier between the liquids, in which case the liquids may be immiscible or miscible. As used herein, the term “thin” refers to a membrane with a width of 2 to 100 nm. In some embodiments, the lipid membrane may comprise a lipid bilayer. In some embodiments, the polymer membrane may comprise a synthetically created membrane capable of enacting a driving force (e.g. pressure or concentration gradients) on particles on either side of the polymer membrane.

As used herein, “air” may refer to a gas or mixture of gasses, such as atmospheric air, oxygen, nitrogen, helium, neon, argon, an inert gas, or a reactive gas.

As used herein, “bulk flow” may refer to movement of objects or fluid down a pressure gradient or temperature gradient of substances in bulk or in masses.

As used herein, “microvortex” may refer to small vortices generated in microfluidic platforms by an acoustic microstreaming process.

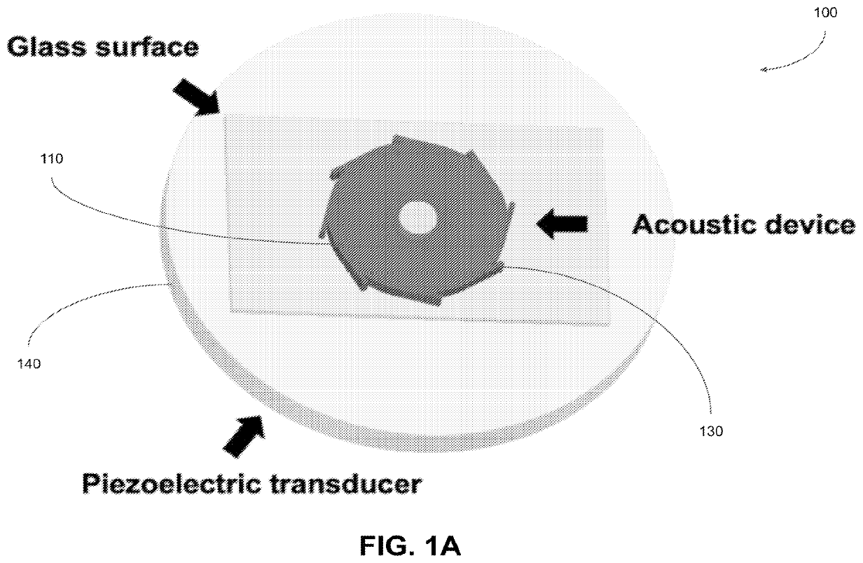

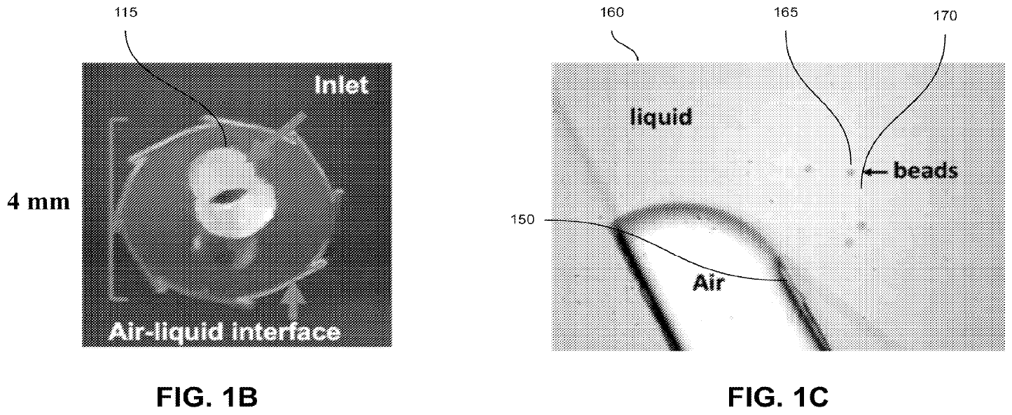

Referring now to FIGS. 1A-1C, the present invention features a microfluidic platform (100) for measuring fluid viscosity. In some embodiments, the microfluidic platform (100) may comprise a main chamber (110). The main chamber (110) may comprise an inlet (115). The microfluidic platform may further comprise one or more cavity acoustic transducers (CATs) (130). The one or more CATs (130) may be dead-end channels coupled to the main chamber (110). A configuration of the CATs (130) may be positioned lateral to the main chamber (110), above the main chamber (110), below the main chamber (110), or a combination thereof. The microfluidic platform (100) may further comprise an external acoustic source (140) coupled to the main chamber (110). In some embodiments, the external acoustic source (140) may comprise a piezoelectric transducer (PZT). The microfluidic platform (100) may further comprise a fluid (160) disposed through the inlet (115) to the main chamber (110). Said fluid (160) may comprise one or more beads (165). The fluid (160) may intersect the CATs (130) to form one or more interfaces (150). In some embodiments, the one or more interfaces (150) may comprise a gas-liquid interface, a liquid-liquid interface, a lipid membrane, a polymer membrane, a nano-particle membrane, or a combination thereof.

The CATs (130) may be configured to oscillate, by the external acoustic source (140), the one or more interfaces (150) to generate microstreaming flow patterns (170) trapping the one or more beads (165) therein. A viscosity of the fluid (160) can be derived from the velocity of the one or more beads (165) in the microstreaming flow patterns (170). In some embodiments, the microfluidic platform (100) may further comprise a plurality of additional chambers, each additional chamber may comprise a corresponding inlet. The plurality of additional chambers may not be fluidly connected to each other or to the main chamber (110). This may allow for the processing of many samples at one time. In some embodiments, the main chamber (110) may comprise an outlet for extracting the fluid (160). The microstreaming flow patterns (170) may comprise bulk flow for direct flow velocity measurement or one or more microvortices.

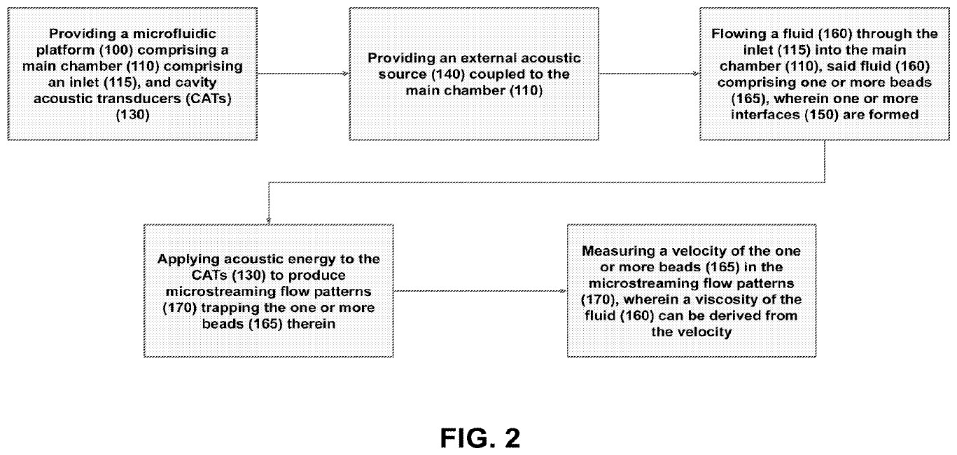

Referring now to FIG. 2, the present invention features a method for measuring fluid viscosity. In some embodiments, the method may comprise providing a microfluidic platform (100). The microfluidic platform (100) may comprise a main chamber (110) which may comprise an inlet (115). The microfluidic platform (100) may further comprise one or more cavity acoustic transducers (CATs) (130). The one or more CATs (130) may be dead-end channels coupled to the main chamber (110). A configuration of the CATs (130) may be positioned lateral to the main chamber (110), above the main chamber (110), below the main chamber (110), or a combination thereof. The method may further comprise providing an external acoustic source (140) coupled to the main chamber (110). The external acoustic source (140) may comprise a piezoelectric transducer (PZT). The method may further comprise flowing a fluid (160) through the inlet (115) into the main chamber (110). Said fluid (160) may comprise one or more beads (165). The fluid (160) may intersect the CATs (130) to form one or more interfaces (150). The one or more interfaces (150) may comprise a gas-liquid interface, a liquid-liquid interface, a lipid membrane, a polymer membrane, a nano-particle membrane, or a combination thereof. The method may further comprise applying acoustic energy to the CATs (130) via the external acoustic source (140) to oscillate, by the external acoustic source (140), the one or more interfaces (150). Oscillating the one or more interfaces (150) produces microstreaming flow patterns (170) trapping the one or more beads (165) therein. The method may further comprise measuring a velocity of the one or more beads (165) in the microstreaming flow patterns (170). A viscosity of the fluid (160) can be derived from the velocity. Velocity is inversely proportional to viscosity in fluids, and thus a higher measured velocity value results in a low viscosity value. The calculation for finding viscosity is as follows:

$$η = \frac{2 {ga}^{2} ( Δρ )}{9 v} ;$$

wherein Δρ is the density difference between the fluid and a bead, a is the radius of the bead, g is the acceleration due to gravity, and v is the measured velocity of the bead in the microvortices.

In some embodiments, the microfluidic platform (100) may further comprise a plurality of additional chambers, each additional chamber may comprise a corresponding inlet. The plurality of additional chambers may not be fluidly connected to each other or to the main chamber (110). This may allow for the processing of many samples at one time. In some embodiments, the main chamber (110) may comprise an outlet for extracting the fluid (160). The microstreaming flow patterns (170) may comprise bulk flow for direct flow velocity measurement or one or more microvortices.

The present invention features a microfluidic viscometer platform that utilized Lateral Cavity Acoustic Transducers (LCATs): The device has a laterally embedded microbubble that forms air-liquid interfaces and can be actuated by a piezoelectric transducer placed below the chip (FIG. 1A). The microfluidic viscometer is designed to be a well plate shape that allows direct loading of the fluid sample at the inlet (FIG. 1B).

The air-liquid interface generates acoustic microstreaming that traps one or more beads within, and the speed of one or more beads will be a direct indicator of fluidic viscosities (FIG. 1C). The maximum speed of the one or more beads will occur at the air-liquid interface and is measured within 3 seconds.

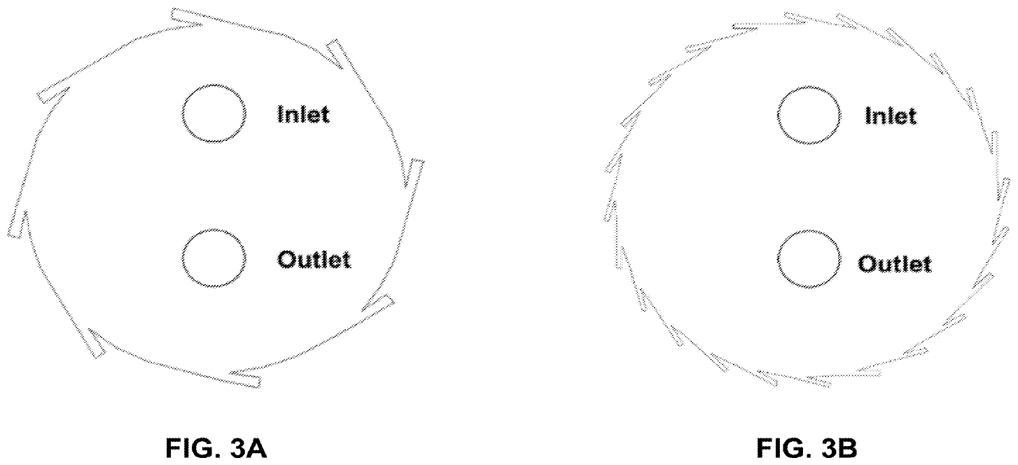

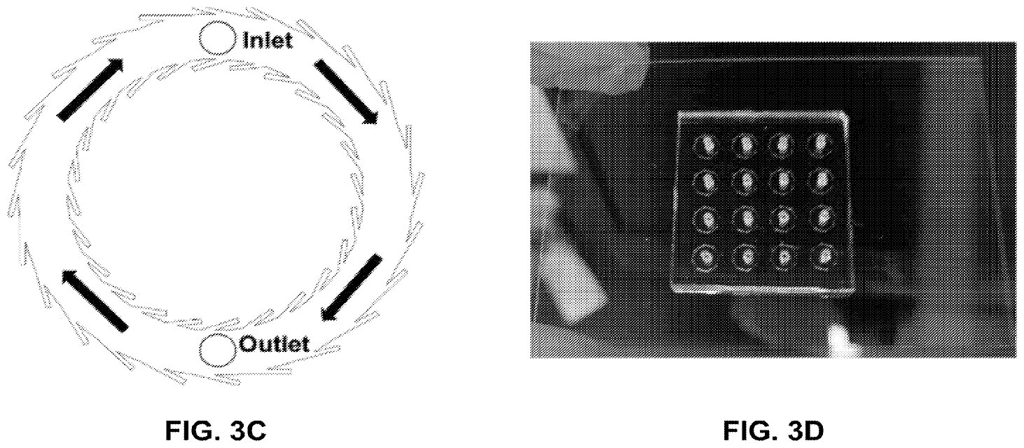

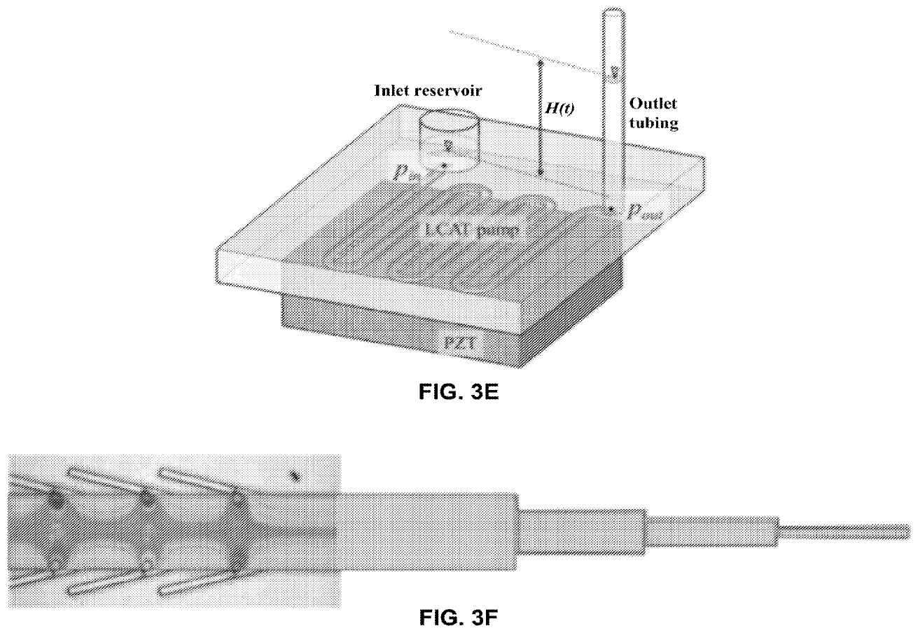

The present invention features several different configurations with an 8-tip design and a scaled-up 24-tip design which allow direct measurement of the microstreaming at the air-liquid interface (FIGS. 3A-3B). Furthermore, the present invention features a viscometer that can induce bulk flow for direct flow velocity measurement without the need to observe an air-liquid interface which requires a zoomed-in magnification and can be observed with the human eye and is user-friendly (FIG. 3C). The proposed design can be manufactured as arrays to have either 16 or 96 wells for high throughput measurement for multiple samples (FIG. 3D). Furthermore, bulk flow can be read via height or a laddered capillary burst pressure (FIGS. 3E-3F).

An exemplary method of use for the present invention may be a rapid measurement of antibody solutions with low volume and high speed and accuracy. Antibodies and protein manufacturing processes are essential for antibody therapies to treat cancer and other infectious diseases. The viscosity of these antibodies is an important indicator for body injection as high viscous fluid can be detrimental to a patients overall survival. The ability to screen millions of antibodies and proteins and select functional products is of particular interest in the pharmaceutical industry. For instance, early screening of viscosities of monoclonal antibodies requires precise measurement of viscosities to select the most optimal antibodies for therapeutic injection. Despite the existence of instruments to conduct measurements, there is still a great need for rapid, accurate, and low sample volume consumption methods for protein screening.

The present invention features a method of rapidly measuring a viscosity of a small volume of an antibody solution. The method may comprise providing a microfluidic platform (100) comprising a main chamber (110). The main chamber (110) may comprise an inlet (115). The microfluidic platform (100) may further comprise one or more cavity acoustic transducers (CATs) (130). The one or more CATs (130) may be dead-end channels coupled to the main chamber (110). The method may further comprise providing an external acoustic source (140) coupled to the main chamber (110) and flowing the antibody solution through the inlet (115) into the main chamber (110), The antibody solution may comprise one or more beads (165). The antibody solution may intersect the CATs (130) to form one or more interfaces (150). The method may further comprise applying acoustic energy to the CATs (130) via the external acoustic source (140) to oscillate, by the external acoustic source (140), the one or more interfaces (150). Oscillating the one or more interfaces (150) may produce microstreaming flow patterns (170) trapping the one or more beads (165) therein. The method may further comprise measuring a maximum velocity of the one or more beads (165) at the air-liquid interface in the microstreaming flow patterns (170). The viscosity of the antibody solution may be derived from the velocity. The viscosity of the antibody solution may be used to determine an effectiveness of the antibody solution as a treatment for cancer and infectious diseases.

### Example

The following is a non-limiting example of the present invention. It is to be understood that said example is not intended to limit the present invention in any way. Equivalents or substitutes are within the scope of the present invention.

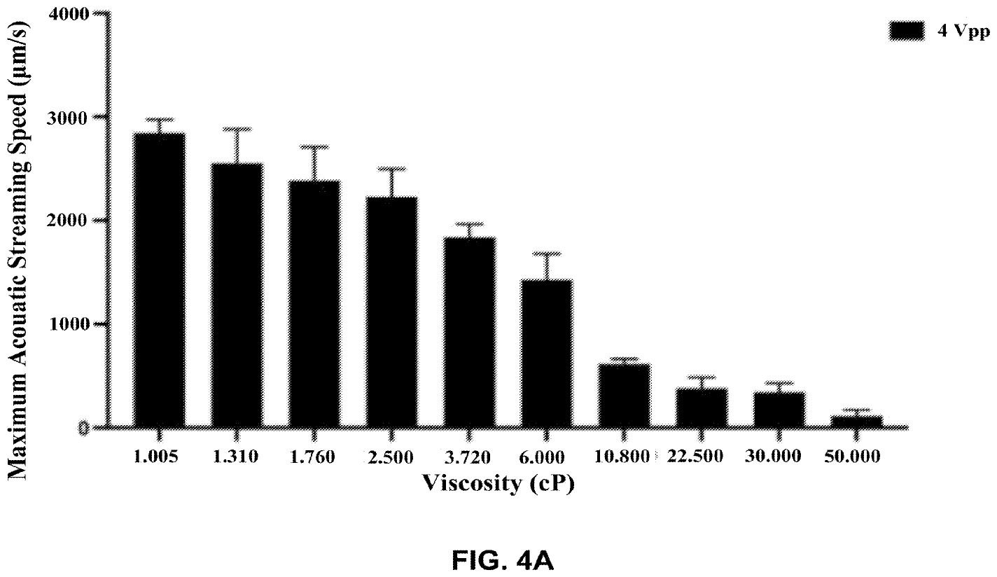

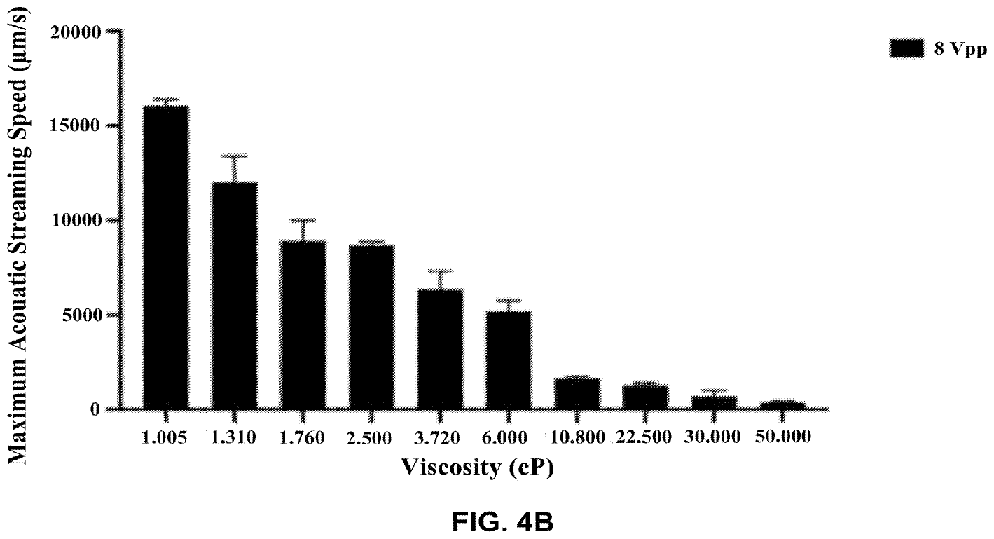

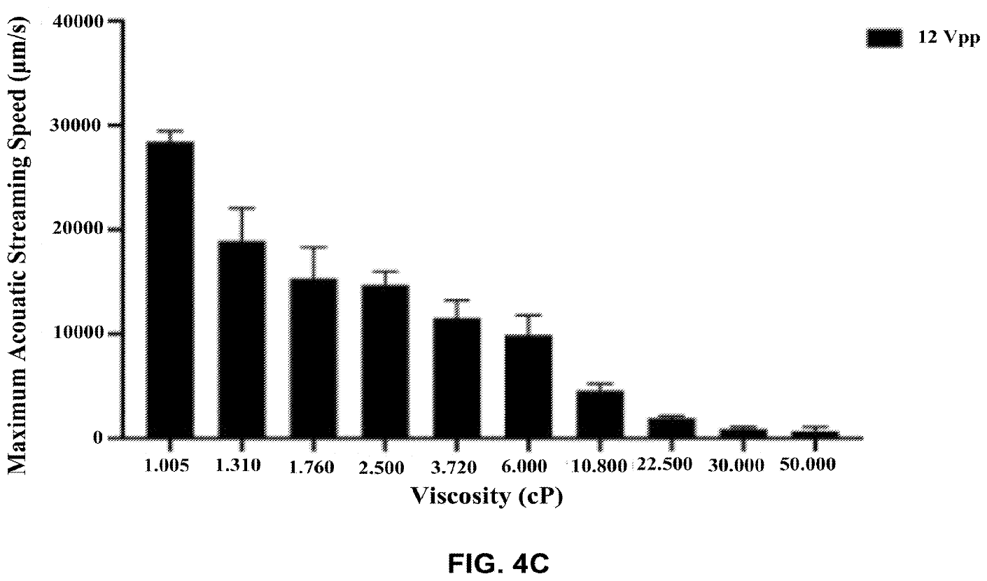

In the present invention, the viscosities of fluid samples were measured by observing the maximum acoustic microstreaming speed and the system had high accuracy to predict fluid viscosities. After beads were trapped within acoustic microstreaming, the speed of the beads was fast and was measured within 3-5 seconds. The beads that were used to validate the present invention were 5 μm and the number of beads was from 3 to 10 beads per vortex to avoid beads interference with the natural flow dynamics. Distinct differences were observed for the maximum acoustic microstreaming speed under different viscosities (FIG. 4A). Furthermore, this was extended to different input power as well. Higher input power from 4 Vₚₚ to 12 Vₚₚ resulted in increased speed of the acoustic microstreaming by 10-fold which allowed even faster viscosity measurement within 1 second (FIGS. 4B-4C).

Although there has been shown and described the preferred embodiment of the present invention, it will be readily apparent to those skilled in the art that modifications may be made thereto which do not exceed the scope of the appended claims.

Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A microfluidic platform (100) for measuring fluid viscosity, comprising:
a. a main chamber (110) comprising an inlet (115);
b. one or more cavity acoustic transducers (CATs) (130), wherein the one or more CATs (130) are dead-end channels coupled to the main chamber (110);
c. an external acoustic source (140) coupled to main chamber (110); and
d. a fluid (160) disposed through the inlet (115) to the main chamber (110), said fluid (160) comprising one or more objects (165), wherein the fluid (160) intersects the CATs (130) to form one or more interfaces (150) capable of generating one or more microstreaming flow patterns (170) when actuated by the external acoustic source (140).

2. The microfluidic platform (100) of claim 1, wherein the external acoustic source (140) comprises a piezoelectric transducer (PZT).

3. The microfluidic platform (100) of claim 1, wherein the CATs (130) are positioned lateral to the main chamber (110), above the main chamber (110), below the main chamber (110), or a combination thereof.

4. The microfluidic platform (100) of claim 1, wherein the one or more interfaces (150) comprise a gas-liquid interface, a liquid-liquid interface, a lipid membrane, a polymer membrane, a nano-particle membrane, or a combination thereof.

5. The microfluidic platform (100) of claim 1, wherein the microfluidic platform (100) further comprises a plurality of additional chambers, each additional chamber comprising a corresponding inlet, wherein the plurality of additional chambers are not fluidly connected to each other or to the main chamber (110).

6. The microfluidic platform (100) of claim 1, wherein the main chamber (110) comprises an outlet for extracting the fluid (160).

7. The microfluidic platform (100) of claim 1, wherein the microstreaming flow patterns (170) comprise bulk flow for direct flow velocity measurement.

8. The microfluidic platform (100) of claim 1, wherein microstreaming flow patterns (170) comprise one or more microvortices.

9. The microfluidic platform (100) of claim 5, wherein the plurality of additional chambers comprises 15 to 95 additional chambers.

10. The microfluidic platform (100) of claim 1, wherein the main chamber (110) comprises a disc shape, wherein the microfluidic platform (100) comprise about 8 to 24 CATs disposed around a circumference of the main chamber (110) and fluidly coupled to the main chamber (110).

11. A method for measuring fluid viscosity comprising:
a. providing a microfluidic platform (100) according to claim 1;
b. flowing a fluid (160) through the inlet (115) into the main chamber (110), said fluid (160) comprising one or more beads (165), wherein the fluid (160) intersects the CATs (130) to form one or more interfaces (150);
c. applying acoustic energy to the CATs (130), via the external acoustic source (140) to oscillate the one or more interfaces (150), wherein oscillating the one or more interfaces (150) produces microstreaming flow patterns (170) trapping the one or more beads (165) therein; and
d. measuring a velocity of the one or more beads (165) in the microstreaming flow patterns (170), wherein a viscosity of the fluid (160) can be derived from the velocity.

12. The method of claim 11, wherein the external acoustic source (140) comprises a piezoelectric transducer (PZT).

13. The method of claim 11, wherein a configuration of the CATs (130) is positioned lateral to the main chamber (110), above the main chamber (110), below the main chamber (110), and a combination thereof.

14. The method of claim 11, wherein the one or more interfaces (150) comprise a gas-liquid interface, a liquid-liquid interface, a lipid membrane, a polymer membrane, a nano-particle membrane, or a combination thereof.

15. The method of claim 11, wherein the microfluidic platform (100) further comprises a plurality of additional chambers, each additional chamber comprising a corresponding inlet, wherein the plurality of additional chambers are not fluidly connected to each other or to the main chamber (110).

16. The method of claim 11, wherein the main chamber (110) comprises an outlet for extracting the fluid (160).

17. The method of claim 11, wherein the microstreaming flow patterns (170) comprise bulk flow for direct flow velocity measurement.

18. The method of claim 11, wherein microstreaming flow patterns (170) comprise one or more microvortices.

19. A method of rapidly measuring a viscosity of an antibody solution, comprising:
a. providing a microfluidic platform (100) comprising a main chamber (110) comprising an inlet (115), and one or more cavity acoustic transducers (CATs) (130), wherein the one or more CATs (130) are dead-end channels coupled to the main chamber (110);
b. providing an external acoustic source (140) coupled to the main chamber (110);
c. flowing the antibody solution through the inlet (115) into the main chamber (110), said antibody solution comprising one or more beads (165), wherein the antibody solution intersects the CATs (130) to form one or more interfaces (150);
d. applying acoustic energy to the CATs (130) via the external acoustic source (140) to oscillate the one or more interfaces (150), wherein oscillating the one or more interfaces (150) produces microstreaming flow patterns (170) trapping the one or more beads (165) therein; and
e. measuring the maximum velocity of the one or more beads (165) at the air-liquid interface in the microstreaming flow patterns (170), wherein the viscosity of the antibody solution can be derived from the velocity;
wherein the viscosity of the antibody solution is used to determine an effectivity of the antibody solution as a treatment for cancer and infectious diseases.

20. A microfluidic platform (100) for measuring fluid viscosity, comprising:
a. a main chamber (110) comprising an inlet (115);
b. one or more cavity acoustic transducers (CATs) (130), wherein the one or more CATs (130) are dead-end channels fluidly coupled to the main chamber (110);
c. an external acoustic source (140) coupled to main chamber (110); and
d. a fluid (160) disposed through the inlet (115) to the main chamber (110), said fluid (160) comprising one or more beads (165), wherein the fluid (160) intersects the CATs (130) to form one or more interfaces (150);
wherein the CATs (130) are configured to oscillate, by the external acoustic source (140), the one or more interfaces (150) to generate microstreaming flow patterns (170) trapping the one or more beads (165) therein, wherein a viscosity of the fluid (160) can be derived from a velocity of the one or more beads (165) in the microstreaming flow patterns (170).
